(19) United States
(12) Patent Application Publication
(10) Pub. No.: US 2021/0244912 A1
(43) Pub. Date: Aug. 12, 2021

(54) INDWELLING URINARY CATHETERS

(71) Applicant: Shai P. PAZ, Jerusalem (IL)

(72) Inventors: Shai P. PAZ, Jerusalem (IL); Yuval KOLODNY, Haifa (IL); Daniel BAR-SELA, Jerusalem (IL); Yaakov NAHMIAS, Mevaseret Zion (IL); Amnon BUXBOIM, Tel Aviv (IL); Yaron BLINDER, Haifa (IL); Yoav MINTZ, Jerusalem (IL); Elchanan FRIED, Jerusalem (IL)

(21) Appl. No.: 17/254,283

(22) PCT Filed: Jun. 20, 2019
(86) PCT No.: PCT/IL2019/050693

§ 371 Date: Dec. 20, 2020

Related U.S. Application Data
(60) Provisional application No. 62/687,400, filed on Jun. 20, 2018.

(51) Int. Cl. A61M 25/00; A61M 25/10

(52) U.S. Cl. CPC A61M 25/0017; A61M 25/0075; A61M 25/06; A61M 2025/0078; A61M 2025/1061; A61M 25/1011

(57) ABSTRACT

Disclosed is an endo-urethral catheter for a male subject. The catheter includes a tube and two balloons. The tube includes a lumen extending there along, an opening(s) on a tube distal section, and a valve positioned in the lumen. A first balloon is disposed about the tube distal section proximally to the opening(s). A second balloon is positioned proximally to the first balloon and distally to the valve. The catheter is configured to be deployed in the urinary system such that the first balloon is positioned in the bladder neck, and the second balloon is positioned adjacently and proximally to the urinary sphincter, thereby anchoring the catheter. The valve is configured to be controllably opened and shut, such as to respectively fluidly couple and decouple the bladder to the proximal end of the tube, thereby allowing the subject to controllably expel urine.

### TECHNICAL FIELD

The present disclosure relates generally to indwelling urinary catheters.

### BACKGROUND

Urinary incontinence—e.g. involuntary leakage of urine or inability to urinate—can result from different types of diseases, injuries, and conditions. In males, these include spinal cord injuries, neurogenic bladder dysfunction, spina bifida, sphincter deficiency, multiple sclerosis, Parkinson's disease, stroke, old age, urethral stricture, bladder spasms, typhoid fever, peritonitis, prostate enlargement, prostate surgery, damage to muscles, and an overactive bladder. Further, in some subjects suffering from urinary incontinence, the urinary sphincter is of itself always closed, so that a catheter must be employed to open the urinary sphincter.

A urethral drainage catheter includes an elastic tube configured to be inserted into the urethra and the bladder. The tube typically includes two lumens running along its length. The larger lumen is used for urine expulsion. In a Foley catheter, the urine expulsion may be substantially continuous with the urine being drained into an external collection bag, which the subject must carry on his person. An external valve, which may be opened and closed by the subject, can be placed on the external collection bag's entry port. The smaller lumen is used to inflate a balloon at a distal section of the catheter which, when the catheter is properly inserted, is positioned in/above the bladder neck and acts to prevent the catheter from slipping out of the body.

Even though many indwelling urinary catheters are provided in various prior art, there still remain several difficulties to overcome in order to avoid complications (e.g. infection, inflammation), injuries, and general discomfort or pain associated with the use of the catheter. In particular, indwelling urinary catheters may include an external portion which protrudes from the tip of the penis and which may cause discomfort/pain and lead to swelling. Moreover, indwelling urinary catheters may require frequent replacement due to infections, coating over time with a biofilm that obstructs the drainage path, and migration of the catheter into the bladder.

There thus exists an unmet need in the art for improved indwelling urinary catheters.

### SUMMARY

Aspects of the disclosure, according to some embodiments thereof, relate to indwelling urinary catheters. More specifically, but not exclusively, aspects of the disclosure, according to some embodiments thereof, relate to indwelling urinary catheters including a valve which may be opened and closed at will, thereby providing a subject with control over urination.

Disclosed herein is a double-balloon indwelling urinary catheter, which is advantageously fully indwelling in the sense that, when properly installed in the urinary system, no part of the catheter is exposed outside the body. The disclosed two balloon design advantageously allows for the catheter to be secured inside the body and prevents slippage of the catheter both outside the body as well as into the bladder. More specifically, when the catheter is properly installed (with the balloons inflated), a first balloon is positioned in the bladder neck and prevents the catheter from slipping out of the body of the subject, while the second balloon is positioned in the urethral tract of the urinary system, adjacently and proximally to the urinary sphincter, such as to advantageously prevent migration (slippage) of the catheter into the bladder. The catheter includes a valve controllably fluidly connecting and disconnecting the distal section of the catheter to the proximal end thereof, which may be actuated by pressing on specific locations on the penis shaft, thereby advantageously allowing the subject to control urination, and dispensing with the need for a urine collection bag (particularly, an external urine collection bag).

A further advantage of the disclosed catheter is that it is easy and safe to deploy and may be self-installed and self-removed by the subject without requiring assistance from a nurse/medical personnel. The disclosure further describes a catheter kit which includes the catheter and an extractor configured for removing the catheter. Advantageously, the extractor is further configured to be used as an applicator in the installation of the catheter.

Thus, according to an aspect of some embodiments, there is provided an endo-urethral catheter for a male subject. The catheter includes an elongated catheter tube, an inflatable first balloon, and an inflatable second balloon. The catheter tube includes:
A urine-expulsion lumen extending along a length of the catheter from a tube proximal end to a tube distal section.
One or more openings positioned on the tube distal section and fluidly connected to the urine-expulsion lumen.
A urine-expulsion valve positioned in the urine-expulsion lumen.

The first balloon is disposed about the tube distal section proximally to the one or more openings. The second balloon is positioned proximally to the first balloon and distally to the urine-expulsion valve. The catheter is configured to be deployed in a urinary system of a male subject with the balloons inflated, such that (i) the one or more openings fluidly couple the urine-expulsion lumen to a bladder of the subject, (ii) the first balloon is positioned in a bladder neck of the subject, and (iii) the second balloon is positioned in a urethral tract of the subject, adjacently and proximally to a urinary sphincter of the subject, so that the two balloons thereby anchor the catheter within the urinary system. The urine-expulsion valve is configured to be controllably opened and shut, such as to respectively fluidly couple and decouple the bladder to the tube proximal end, thereby allowing the subject to controllably expel urine.

According to some embodiments, when the catheter is properly deployed (in the urine system), the tube proximal end is positioned within the urethral tract.

According to some embodiments, he first balloon is configured to prevent the catheter from slipping out of the body of the subject, when the catheter is properly deployed with the first balloon being inflated in the bladder neck.

According to some embodiments, the second balloon is configured to prevent distal migration of the catheter along the urethral tract, when the catheter is properly deployed with the second balloon inflated and positioned in the urethral tract, adjacently and proximally to the urinary sphincter.

According to some embodiments, the urine-expulsion valve is configured (i) to be opened by squeezing thereof, and (ii) to be automatically shut when the squeezing is ceased/sufficiently relaxed, being thereby configured to prevent involuntary leakage of urine.

According to some embodiments, the urine-expulsion valve is configured to function as a one-way valve preventing proximal flow of fluid therethrough.

According to some embodiments, the urine-expulsion valve is a duckbill valve.

According to some embodiments, the urine-expulsion valve is configured to be opened by pressing a penile shaft and/or a glans penis of the subject, such as to squeeze the urine-expulsion valve, when the catheter is properly deployed.

According to some embodiments, the catheter includes at least one inflation lumen extending in parallel to the urine-expulsion lumen in the distal direction from at least one inflation port positioned at the tube proximal end. The at least one inflation lumen is configured to inflate the balloons.

According to some embodiments, each of the at least one inflation lumen includes one or more inflation valves configured to prevent fluid flow in the proximal direction. When open, the one or more inflation valves fluidly couple the balloons to the at least one inflation port. When closed, the one or more inflation valves fluidly decouple the balloons from the at least one inflation port, and thereby act to maintain the balloons inflated.

According to some embodiments, each of the one or more inflation valves is positioned proximally to the urine-expulsion valve.

According to some embodiments, the at least one inflation lumen includes a first inflation lumen and a second inflation lumen extending inside tube walls of the catheter tube on opposite sides of the urine-expulsion lumen.

According to some embodiments, the first inflation lumen is fluidly connected to the first balloon and the second inflation lumen is fluidly connected to the second balloon.

According to some embodiments, the urine-expulsion lumen includes a first inner side-surface on a first section of the tube walls separating the urine-expulsion lumen from the first inflation lumen, and a second inner side-surface on a second section of the tube walls opposite to the first section (i.e. the second section of the tube walls separates the urine-expulsion lumen from the second inflation lumen). The first inner side-surface includes a first protrusion extending laterally therefrom and positioned adjacently to the first inflation valve. The second inner side-surface includes a second protrusion extending laterally therefrom and positioned adjacently to the second inflation valve. The first inflation valve and the first protrusion are configured such as to allow opening the first inflation valve by pressing on the first protrusion. The second inflation valve and the second protrusion are configured such as to allow opening the second inflation valve by pressing on the second protrusion.

According to some embodiments, wherein the protrusions are malleable.

According to some embodiments, each of the first inflation valve and the second inflation valve is configured to function as a one-way valve preventing proximal flow of fluid therethrough.

According to some embodiments, each of the first inflation valve and the second inflation valve is a duckbill valve.

According to some embodiments, the first balloon and the second balloon are fluidly connected.

According to an aspect of some embodiments, there is provided an extractor for removing an endo-urethral catheter deployed in a urine system of a male subject. The extractor includes:
A first handle including a first proximal section and a first distal section which includes a first distal edge.
A second handle including a second proximal section and a second distal section which includes a second distal edge.
A first extension, and second extension, each in the form of an elongated rod.

The handles are configured to allow pressing the proximal sections towards one another and thereby distancing the distal edges from one another. The first extension distally extends from the first distal edge, and the second extension distally extends from the second distal edge. The extractor is configured to be switched between at least two configurations:
A closed configuration, wherein the extensions are disposed alongside one another, such as to contact, or nearly contact, one another.
An open configuration, wherein the proximal sections are pressed towards one another and the distal tip portions of the extensions are spaced apart from one another.

According to some embodiments, in the open configuration the extensions are arranged in a V-like configuration.

According to some embodiments, the two handles are formed of a single piece.

According to some embodiments, the two handles are mechanically coupled to one another, such as to allow switching the extractor between the at least two configurations.

According to some embodiments, the extractor further includes a return-mechanism configured to automatically switch back the extractor from the open configuration to the closed configuration when the pressing of the proximal sections towards one another is ceased or at least sufficiently relaxed.

According to some embodiments, the return mechanism is spring-based.

According to some embodiments, the return-mechanism includes a U-spring clip, wherein the base of the clip extends from the first handle to the second handle.

According to some embodiments, the extractor is configured for use with the catheter described above.

According to an aspect of some embodiments, there is provided a catheter kit including the catheter, described above, and the extractor, described above, a balloon inflation system, and a urine indicator. The balloon inflation system includes (i) a pressable first fluid bag and a first inflation duct configured for fluidly coupling the first fluid bag to the first inflation lumen, and (ii) a pressable second fluid bag and a second inflation duct configured for fluidly coupling the second fluid bag to the second inflation lumen. The urine indicator includes a urine indicator tube fluidly connected to, and extending distally from, a container. The urine indicator tube may be removably attached to the extractor such as to be aligned with the extensions, and to extend in the distal direction beyond the extensions, when the extractor is in the closed configuration.

According got some embodiments, the catheter kit is assembled with the extensions of the extractor, which is in the closed configuration, inserted into the urine-expulsion lumen such that the distal tip portions of the extensions are adjacent to the protrusions on the inner side-surfaces and such that the urine indicator tube extends through the urine-expulsion valve. The inflation ducts of the balloon inflation system are fluidly connected to the inflation ports of the catheter tube.

According to an aspect of some embodiments, there is provided a method for removing the catheter, described above, from a urinary system of a male subject, using the extractor, described above. The catheter is deployed such that the first balloon is inflated and positioned at a bladder neck of the urinary system. The second balloon is inflated and positioned in a urethral tract of the urinary system, adjacently and proximally to a urinary sphincter of the urinary system, such that the balloons secure the catheter within the urinary system. The method includes:
Deploying the extractor, when in the closed configuration, in the catheter, by inserting the extensions of the extractor into the urine-expulsion lumen, such that the distal tip portions of the extensions are adjacent to the protrusions in the urine-expulsion lumen.
Emptying the balloons by switching the extractor to the open configuration, thereby pressing on the protrusions and opening the inflation valves.
Pulling out the proximal section of the catheter tube by pulling out the extractor, and then pulling out the rest of the catheter tube.

According to an aspect of some embodiments, there is provided a method for deploying a catheter in a urinary system of a male subject using the catheter kit described above. The method includes:
Inserting the catheter into a urethral tract of the urine system and gradually pushing the catheter there along until the urine indicator starts draining urine, indicating that the distal section of the catheter tube has reached the bladder, the first balloon is positioned in a bladder neck of the urine system, and the second balloon is positioned in the urethral tract, adjacently and proximally to a urinary sphincter of the urine system.
Inflating the balloons to anchor the catheter by squeezing the fluid bags of the balloon inflation system, so that fluid ejected from the fluid bags forces open the inflation valves in the inflation lumens, distally flows therethrough, and inflates the balloons.
Removing the extractor, and thereby the urine indicator, and removing the balloon inflation system.

According to some embodiments, in the step of inflating the balloons, the first balloon is inflated prior to inflating the second balloon.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A presents a schematic, perspective view of a double-balloon, indwelling urinary catheter with the two balloons inflated, according to some embodiments;

FIG. 1B presents a schematic side-view of the catheter of FIG. 1A, according to some embodiments;

FIG. 1C presents a schematic, cross-sectional view of the catheter of FIG. 1A, according to some embodiments;

FIG. 1D presents a schematic, close-up view of FIG. 1A, according to some embodiments;

FIG. 1E presents a schematic, close-up view of FIG. 1A, according to some embodiments;

FIG. 2A presents a schematic, perspective view of an extractor configured to remove the catheter of FIG. 1A from a urethral tract of a male subject into which the catheter has been inserted, the extractor being depicted in a closed configuration, according to some embodiments;

FIG. 2B presents a schematic, perspective view of the extractor of FIG. 2A in an open configuration, according to some embodiments;

FIG. 3A presents a schematic, perspective view of a catheter kit for insertion of the catheter of FIG. 1A, according to some embodiments;

FIG. 3B presents a schematic, exploded view of the catheter kit of FIG. 3A, according to some embodiments;

FIG. 4 presents a flowchart of a method for removing an indwelling urine catheter, such as the catheter of FIG. 1A, from the urethral tract of a male subject, using an extractor such as the extractor of FIG. 2A, according to some embodiments;

FIGS. 5A-5D schematically depict stages in the removal of the catheter of FIG. 1A from the urethral tract of a male subject, using the extractor of FIG. 2A and in accordance with the method of FIG. 4, according to some embodiments;

FIG. 6 presents a flowchart of a method for anchoring (securing) an indwelling urine catheter, such as the catheter of FIG. 1A, in the urethral tract of a male subject, using a catheter kit such as the catheter kit of FIG. 3A, according to some embodiments; and

FIGS. 7A-7E schematically depict stages in the anchoring (securing) of the catheter of FIG. 1A in the urethral tract of a male subject using the catheter kit of FIG. 3A and in accordance with the method of FIG. 6, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

As used herein, according to some embodiments, a “proximal” end/section/portion/tip of a medical element/component/device may refer to a part of the element/component/device that is closer (e.g. during use/insertion/extraction of the device) to the hand(s) of a user (e.g. a medical practitioner inserting a catheter, a subject removing the catheter), operating/manipulating the element/component/device, as compared to at least one other part of the element/component/device. Similarly, according to some embodiments, a “distal” end/section/portion/tip of a medical element/component/device may refer to a part of the element/component/device that is farther from the hand(s) of a user, operating/manipulating the element/component/device, as compared to at least one other part of the element/component/device. According to some embodiments, a “distal” end/section/portion/tip of an element/component/device may refer to a part of the element/component/device that is closer to a diagnosis or treatment site in the body of a patient as compared to at least one other part of the element/component/device.

As used herein, according to some embodiments, the term “lateral” is used to denote a direction which is perpendicular to a “distal” direction and a “proximal” direction (which points opposite to the distal direction).

To facilitate the description, in figures provided with a coordinate system, the distal direction may be set to coincide with the direction of the (positive) z-axis.

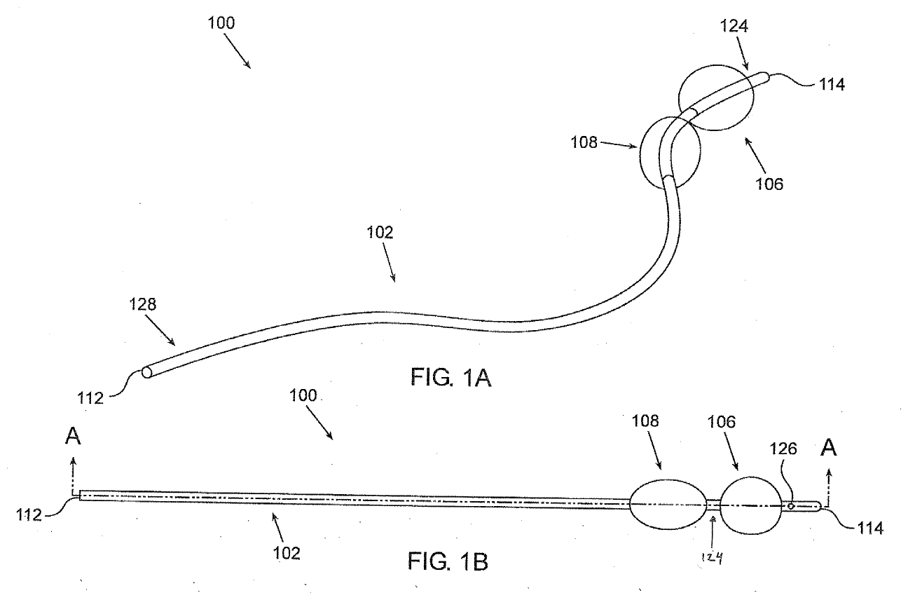

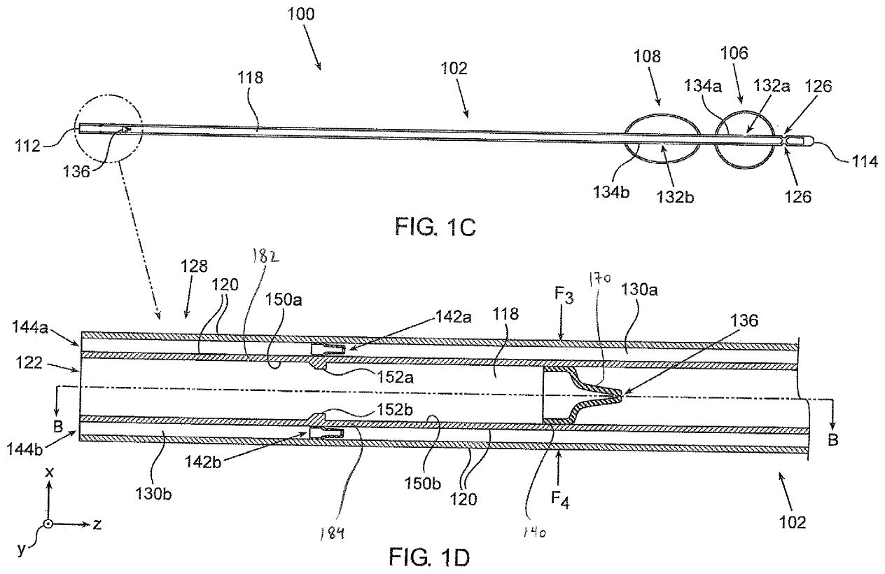

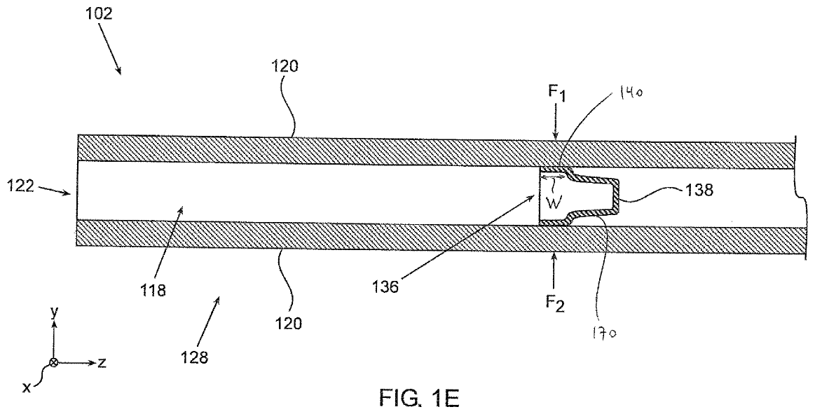

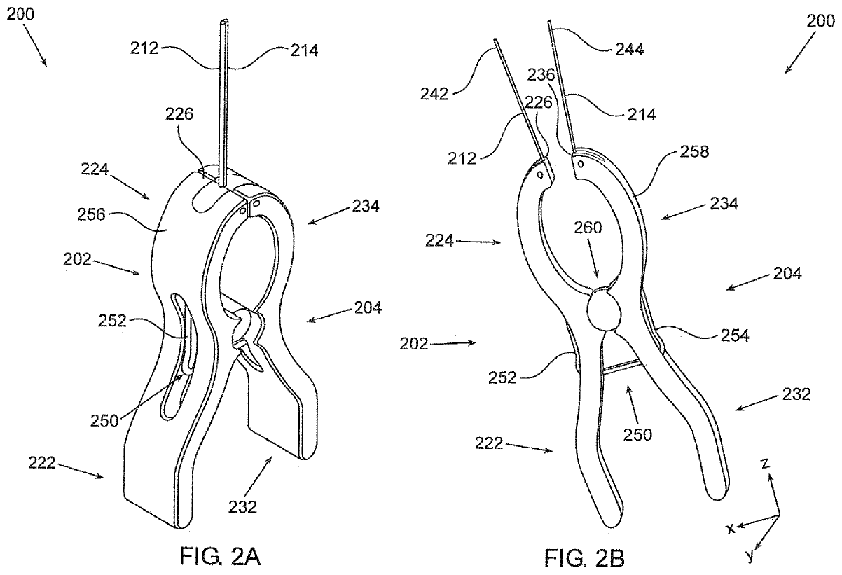

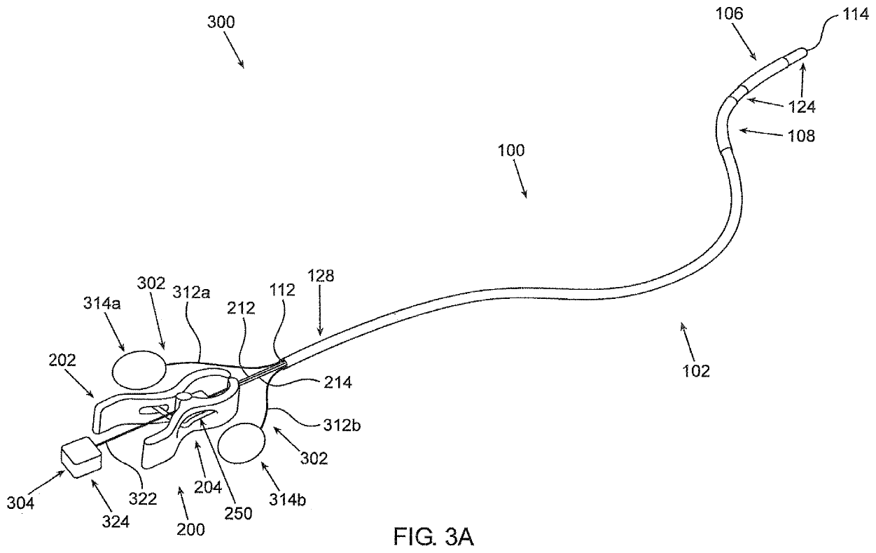

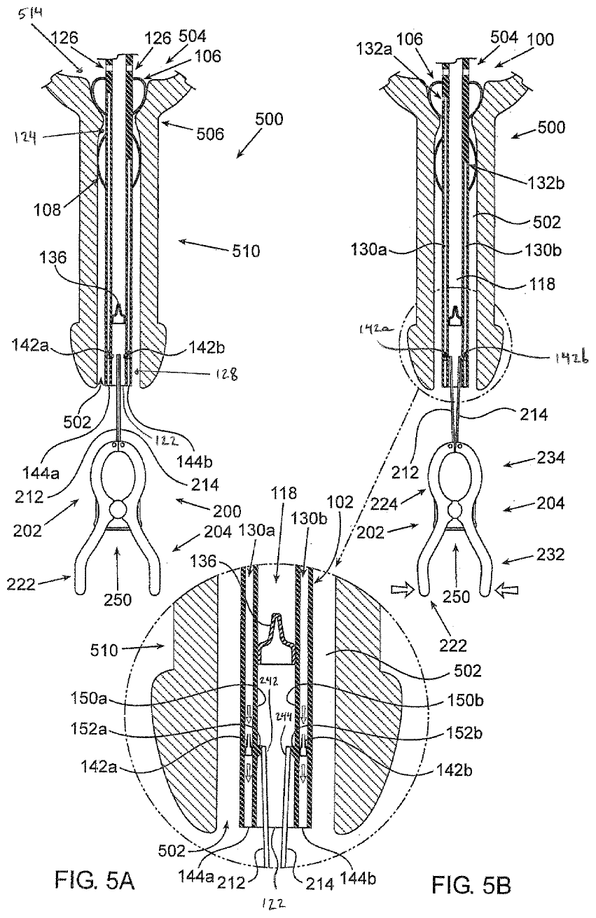

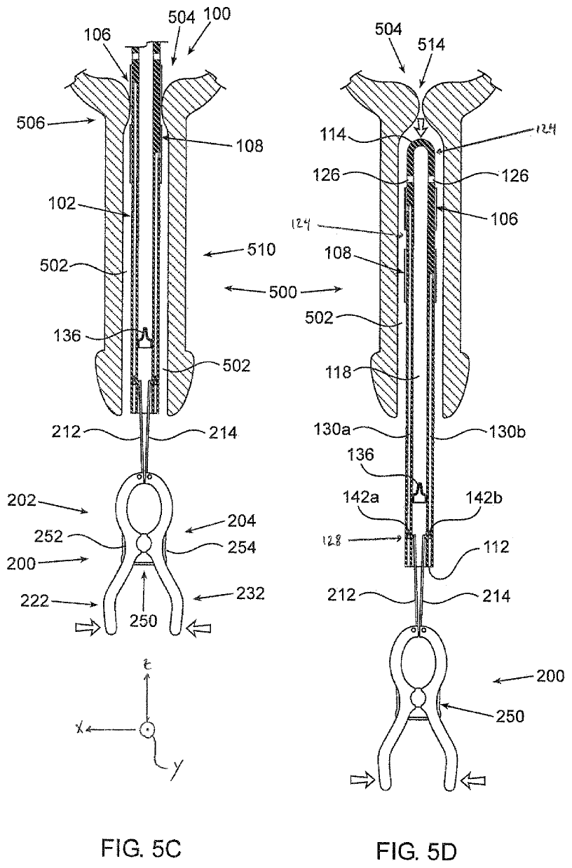

According to an aspect of some embodiments, and as schematically depicted in FIGS. 1A-1E, there is provided a double-balloon, indwelling urinary catheter 100 (i.e. endo-urethral catheter including two balloons). Catheter 100 includes an elongated and flexible catheter tube 102, an inflatable first balloon 106, and an inflatable second balloon 108. FIG. 1A presents a perspective view of catheter 100 with balloons 106 and 108 inflated (shown deflated, for example, in FIG. 3B), according to some embodiments. FIG. 1B presents a side-view of catheter 100, according to some embodiments. FIG. 1C presents a cross-sectional view of catheter 100 taken along a line A-A (shown in FIG. 1B), according to some embodiments.

Catheter tube 102 extends from a tube proximal end 112 (i.e. the proximal end of catheter tube 102) to a tube distal end 114 (i.e. the distal end of catheter tube 102). Catheter tube 102 includes a urine-expulsion lumen 118 extending along the length thereof. Urine-expulsion lumen 118 extends within catheter tube 102, along the length thereof, from a urine-expulsion port 122, positioned at tube proximal end 112, to a tube distal section 124 (i.e. a distal section of catheter tube 102 which includes, and terminates at, tube distal end 114). Tube distal section 124 includes one or more openings 126 (for example, two openings as depicted in the figures). According to some embodiments, not depicted in the figures, tube distal end 114 is open and defines one of openings 126 (or all the openings when one or more openings 126 consist of a single opening). Urine-expulsion lumen 118 fluidly couples urine-expulsion port 122 to openings 126, which in turn fluidly couple urine-expulsion lumen 118 to the exterior of catheter 100.

First balloon 106 and second balloon 108 are disposed about catheter tube 102. According to some embodiments, first balloon 106 is disposed about tube distal section 124 proximally to openings 126. Second balloon 108 may also be disposed about tube distal section 124 and is positioned proximally to first balloon 106.

Referring also to FIG. 1D, FIG. 1D is a partial, closeup view of FIG. 1B. More specifically, FIG. 1D presents a cross-sectional view of a tube proximal section 128 (i.e. a proximal section of catheter tube 102) taken along a line A-A, according to some embodiments. Catheter tube 102 further includes one or more inflation lumens 130 (for example, a pair of side-lumens, as depicted in the figures) extending within tube walls 120 of catheter tube 102 in parallel to urine-expulsion lumen 118. Inflation lumens 130 may be used to inflate balloons 106 and 108, as elaborated on below. According to some embodiments, and as depicted in the figures, a first inflation lumen 130a (from inflation lumens 130) extends from tube proximal end 112 to first balloon 106, and is fluidly decoupled from second balloon 108, and a second inflation lumen 130b (from inflation lumens 130) extends from tube proximal end 112 to second balloon 108 (and is fluidly decoupled from first balloon 106). More specifically, first inflation lumen 130a may extend in the distal direction until a first inflation aperture 132a on an outer wall 134a of first inflation lumen 130a, and second inflation lumen 130b may extend in the distal direction until a second inflation aperture 132b on an outer wall 134b of second inflation lumen 130b. First inflation aperture 132a may be positioned adjacently to first balloon 106 and is configured for inflating first balloon 106. Second inflation aperture 132b may be positioned adjacently to second balloon 108 (and therefore proximally to first inflation aperture 132a) and is configured for inflating second balloon 108.

According to some alternative embodiments, first balloon 106 and second balloon 108 are fluidly coupled. According to some such embodiments, each of the inflation lumens extends from tube proximal end 112 to second balloon 108. Alternatively, catheter tube 102 may include only a single inflation lumen extending from tube proximal end 112 to second balloon 108.

Also indicated is a line B-B centrally extending through catheter tube 102.

Referring also to FIG. 1E, FIG. 1E is a partial close up view of catheter tube 102, according to some embodiments. The presented view is a cross-sectional view taken along line B-B of FIG. 1D. Urine-expulsion lumen 118 includes a urine-expulsion valve 136. In an open state, urine-expulsion valve 136 fluidly couples urine-expulsion port 122 and openings 126. In a closed state (i.e. when shut), urine expulsion valve 136 fluidly decouples openings 126 from urine-expulsion port 122. Urine-expulsion valve 136 is controllably switchable between the open state and the closed state, as elaborated on below. Urine-expulsion valve 136 is further configured to function as a one-way valve in the sense of preventing proximal flow (i.e. flow in an opposite sense to the direction defined by the (positive) z-axis) of fluid therethrough (and thereby prevents unwanted leakage of urine). More specifically, urine-expulsion valve 136 is configured such as to not open as a result of fluid pressure on the distal side thereof (at least at pressures up to those typical of urine in a bladder when the bladder is full). According to some embodiments, and as depicted in the figures, urine-expulsion valve 136 is a duckbill valve mounted within urine-expulsion lumen 118 such that a flattened end 138 thereof (i.e. the duckbill or “beak”) points in the distal direction (and thereby preventing proximal flow of fluid therethrough). Also indicated is a circumferential base 140 of urine-expulsion valve, a width W of circumferential base 140, and a beak 170. In such embodiments, urine-expulsion valve 136 may be opened by the squeezing thereof. More specifically, the duckbill valve may be opened by applying opposite lateral forces thereon (e.g. on circumferential base 140), as indicated by arrows F₁ and F₂ in FIG. 1E. According to some embodiments, the duckbill valve may be resilient in the sense of automatically shutting when the squeezing thereof is ceased/sufficiently relaxed. According to some embodiments, to shut the duckbill valve, the duckbill valve must be squeezed by applying opposite lateral forces thereon at 90° to the forces that must be applied to open the duckbill, as indicated by arrows F₃ and F₄ in FIG. 1D.

Catheter tube 102 further includes one or more inflation valves 142 positioned in one or more inflation lumens 130, respectively. According to some embodiments, and as depicted in the figures, inflation valves 142 include a first inflation valve 142a, positioned in first inflation lumen 130a proximally to urine-expulsion valve 136, and a second inflation valve 142b, positioned in second inflation lumen 130b opposite to first inflation valve 142a. Each of inflation valves 142 is switchable between an open configuration and a closed configuration. When both inflation valves 142 are open, first balloon 106 and second balloon 108 are fluidly coupled via inflation lumens 130 to tube proximal end 112, such as to allow their inflation by injecting fluid into inflation lumens 130. According to some embodiments, and as depicted in the figures, first inflation valve 142a is configured to controllably fluidly couple and decouple first balloon 106 to a first inflation port 144a, positioned at (or near) tube proximal end 112, such as to allow inflation thereof with fluid injected via first inflation port 144a. Similarly, according to some embodiments, and as depicted in the figures, second inflation valve 142b is configured to controllably fluidly couple and decouple second balloon 108 to a second inflation port 144b, positioned at (or near) tube proximal end 112, such as to allow inflation thereof with fluid injected via second inflation port 144b.

Similarly to urine-expulsion lumen 118, each of inflation valves 142 is further configured to function as a one-way valve in the sense of preventing proximal flow of fluid therethrough (thereby preventing escape of fluid from balloons 106 and 108 when inflated). More specifically, and as elaborated on below, each of inflation valves 142 is configured such as to not open as a result of fluid pressure on the distal side thereof when balloons 106 and 108 are fully inflated. According to some such embodiments, each of inflation valves 142 is a duckbill valve, positioned within the respective inflation lumen such that the respective flattened end thereof points in the distal direction (i.e. in the direction defined by the (positive) z-axis).

According to some embodiments, and as depicted in the figures, a first inner side-surface 150a of tube walls 120 may include at least one first protrusion 152a (e.g. a bulge) projecting inwardly therefrom (i.e. towards the line B-B in FIG. 1D). Similarly, a second inner side-surface 150b of tube walls 120, which is positioned opposite to first inner side-surface 150a, may include at least one second protrusion 152b projecting inwardly therefrom. More specifically, first inner side-surface 150a forms an inner surface of urine-expulsion lumen 118 on a first section 182 of tube walls 120 which separates urine-expulsion lumen 118 from first inflation lumen 130a. Similarly, second inner side-surface 150b forms an inner surface of urine-expulsion lumen 118 on a second section 184 of tube walls 120 which separates urine-expulsion lumen 118 from second inflation lumen 130b. First protrusion 152a is positioned on first inner side-surface 150a adjacently to first inflation valve 142a (i.e. at the same “height” as measured with respect to the z-axis), and second protrusion 152b is positioned on second inner side-surface 150b adjacently to second inflation valve 142b. According to some embodiments, protrusions 152 may exhibit plasticity (e.g. be malleable), as elaborated on below. According to some embodiments, protrusions 152 (i.e. protrusions 152a and 152b) may be configured to essentially function as knobs for opening inflation valves 142, as elaborated on below.

According to some embodiments, the distance between first balloon 106 and second balloon 108 is between about 2.5 cm and about 5 cm. According to some embodiments, the length of second balloon 108 is between about 2 cm and about 7 cm. According to some embodiments, second balloon 108 radially (i.e. outwardly laterally) projects between about 8 mm and about 20 mm from catheter tube 102.

According to some embodiments, catheter tube 102 may be made of a material including rubber and/or silicone, and balloons 106 and 108 may be made of a material including rubber and/or silicone, as known in the art of urinary indwelling catheters. Similarly, according to some embodiments, urine-expulsion valve 136 and inflation valves 142 may be made of a material including rubber and/or silicone.

According to an aspect of some embodiments, not depicted in the figures, there is provided a urinary indwelling catheter. The catheter is similar to catheter 100 but differs therefrom in including a plurality of longitudinally extending balloons in place of second balloon 108. That is, when the catheter is deployed in the urine system of a male subject with the first balloon inflated and positioned in the bladder neck, the plurality of balloons are positioned adjacently and proximally to the urinary sphincter, such as to anchor the catheter and prevent distal migration of the catheter. According to some such embodiments, when inflated, the elongated balloons are spaced apart, thereby potentially preventing damage to the epithelial cell layer surrounding the urinary sphincter.

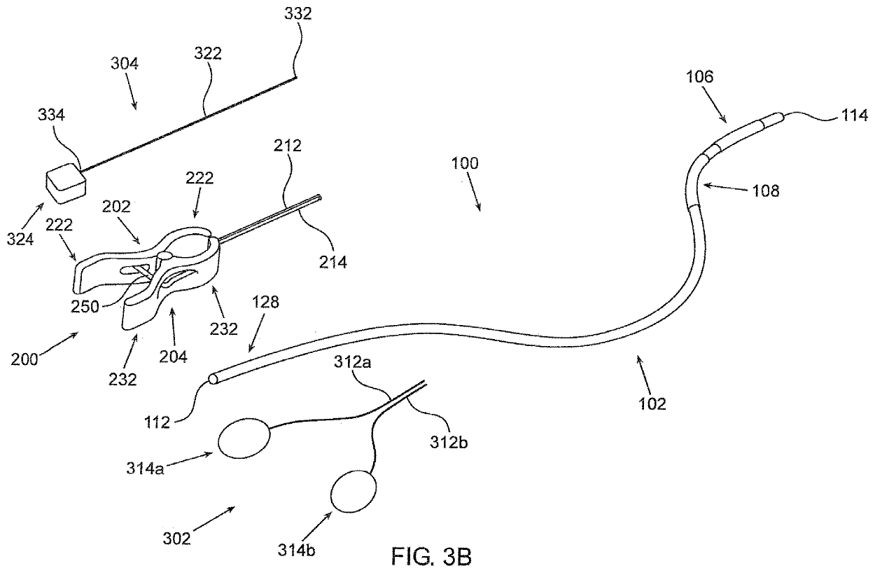

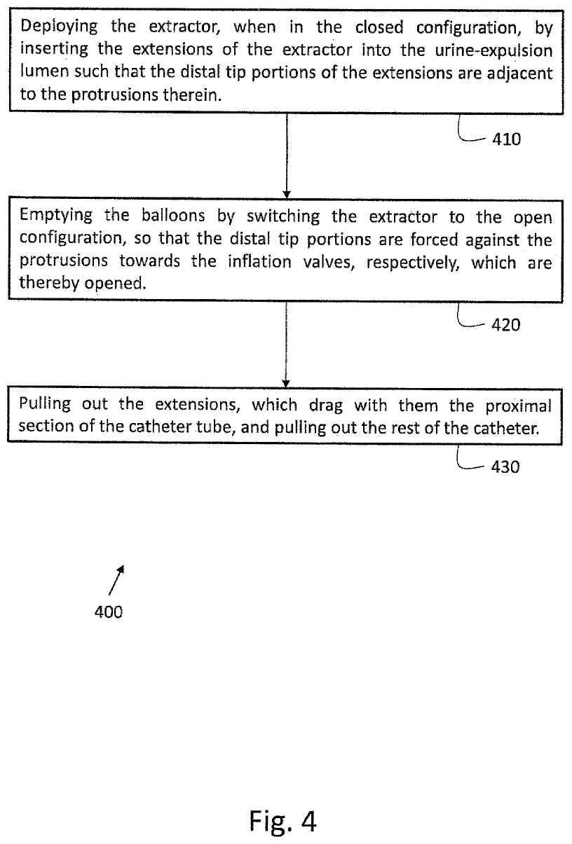

According to an aspect of some embodiments, and as depicted in FIGS. 2A and 2B, there is provided an extractor 200. FIG. 2A schematically depicts extractor 200 in a first (closed) configuration, according to some embodiments. FIG. 2B schematically depicts extractor 200 in a second (open) configuration. As described below, extractor 200 is controllably switchable between the first and second configurations. Extractor 200 includes a first handle 202, a second handle 204, an elongated first extension 212, and an elongated second extension 214. First handle 202 includes a first handle proximal (bottom) section 222 and a first handle distal (upper) section 224 terminating in a first handle distal edge 226. First extension 212 extends in the distal direction from first handle distal edge 226. Similarly, second handle 204 includes a second handle proximal section 232 and a second handle distal section 234 terminating in a second handle distal edge 236. Second extension 214 extends in the distal direction from second handle distal edge 236.

According to some embodiments, and as depicted in the figures, each of extensions 212 and 214 is shaped as a thin rod. According to some such embodiments, each of extensions 212 and 214 is straight or substantially straight. First extension 212 terminates in the distal direction in a first extension distal tip portion 242 and second extension 214 terminates in the distal direction in a second extension distal tip portion 244. Also indicated is a first handle side-surface 256 and a second handle side-surface 258 facing laterally and away from extractor 200 (i.e. opposite and along the direction defined by the x-axis, respectively).

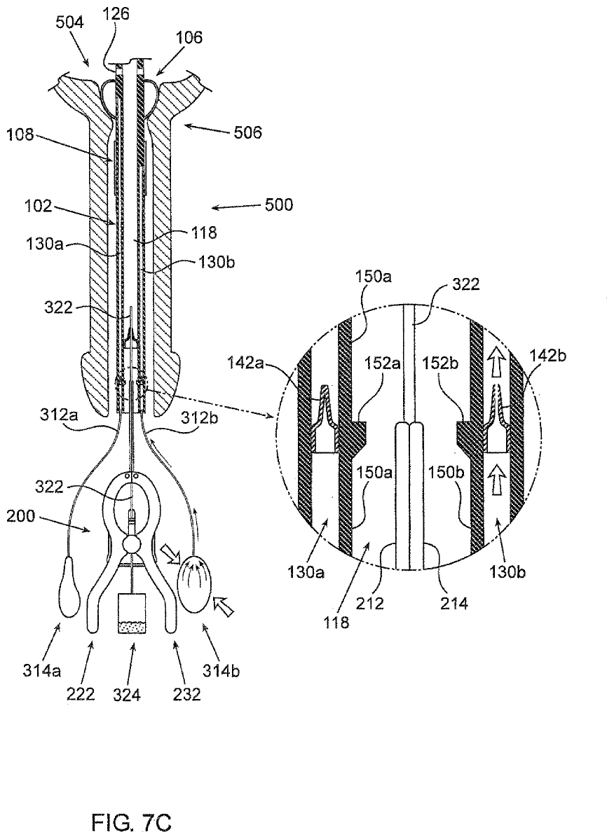

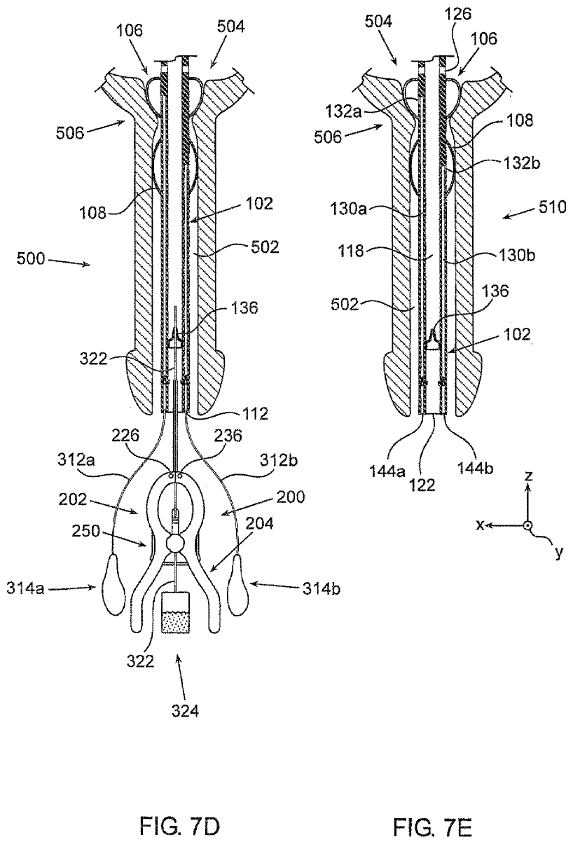

According to some embodiments, not depicted in the figures, one or more of extensions 212 and 214 may be slightly curved. According to some embodiments, each of first extension distal tip portion 242 and second extension distal tip portion 244 may be laterally outwardly curved or bent at the tip, such as to potentially facilitate a better grip of protrusions 152a and 152b when dragging catheter out of the urethral tract as described below in the description of FIGS. 6-7E.

First handle 202 and second handle 204 may be axially associated, as a non-limiting example, in a similar manner to the association between the two prongs of a clothespin. According to some embodiments, extractor 200 includes an axis member 260 configured to provide the axial association between first handle 202 and second handle 204. According to some embodiments, first handle 202 and second handle 204 are mechanically coupled by a return-mechanism configured to resist the pressing of handles 202 and 204 towards one another and to return extractor 200 to the first configuration from the second configuration (once the pressing of handles 202 and 204 is ceased). According to some embodiments, the return-mechanism may be or include a spring-based return mechanism, a hydraulic-based return mechanism, and the like. As a non-limiting example and as depicted in the figures, the spring-mechanism may be, or include, a spring clip 250 (e.g. a U-spring clip with a first arm 252 thereof installed in first handle 202 and a second arm 254 thereof installed in second handle 204). According to some embodiments, in the first configuration, spring clip 250 may be relaxed, while in the second configuration, spring clip 250 is compressed.

Handle proximal sections 222 and 232 are configured to be pressed towards one another using the fingers (e.g. the thumb and index finger of one of the hands). To switch from the first configuration to the second configuration, handle proximal sections 222 and 232 are pressed towards one another, thereby compressing spring clip 250. To switch back from the second configuration to the first configuration, the pressing of handle proximal sections 222 and 232 is relaxed (e.g. handle proximal sections 222 and 232 are no longer forced towards one another), so that the opposite lateral forces, exerted by spring clip 250 on first handle proximal section 222 and second handle proximal section 232, push handle proximal sections 222 and 232 away from one another.

In the first configuration, first handle distal edge 226 and second handle distal edge 236 may be adjacent, such as to be in contact with one another, or nearly in contact with one another. Accordingly, first extension 212 and second extension 214 may be adjacently aligned, such as to be in contact, or nearly in contact with one another (along the lengths thereof). As handle proximal sections 222 and 232 are pressed towards one another, handle distal edges 226 and 236 are moved apart from one another. Consequently, extensions 212 and 214 are also moved apart from one another, so that, according to some embodiments, extension distal tip portions 242 and 244 are moved apart the farthest.

According to some embodiments, first extension 212 and second extension 214 have the same, or substantially the same, dimensions. More specifically, first extension 212 and second extension 214 are dimensioned such as to be insertable into tube proximal section 128 (via urine-expulsion port 122), when extractor 200 is in the first configuration. According to some embodiments, the length(s) of extensions 212 and 214 may be such that, when fully inserted into catheter tube 102, extension distal tip portions 242 and 244 are positioned proximally to urine-expulsion valve 136 and do not come into contact with urine-expulsion valve 136, thereby preventing potential damage to urine-expulsion valve 136 during the insertion of extensions 212 and 214 into urine-expulsion lumen 118 (described below). Further, the dimensions of first extension 212 and second extension 214 are such as to allow switching extractor 200 from the first configuration to the second configuration when extensions 212 and 214 are both inserted into catheter tube 102, as further elaborated on below.

According to some embodiments, handles 202 and 204 may be made of a polymeric material, such as acrylonitrile butadiene styrene (ABS) plastic. According to some embodiments, extensions 212 and 214 may be made of stainless steel.

According to an aspect of some embodiments, and as depicted in FIGS. 3A and 3B, there is provided a catheter kit 300, which includes catheter 100 and which is configured for deployment of catheter 100 in the urinary system of a male subject and subsequent removal of catheter 100. FIG. 3A presents a schematic, perspective view of catheter kit 300, according to some embodiments. FIG. 3B presents a schematic, exploded view of catheter kit 300, according to some embodiments. In addition to catheter 100, catheter kit 300 may include extractor 200 (or an extractor or similar thereto), a balloon inflation system 302, and a urine indicator 304. In both FIGS. 3A and 3B, balloons 106 and 108 are shown deflated.

Balloon inflation system 302 includes one or more inflation ducts 312 and one or more pressable fluid bags 314 fluidly connected to one or more inflation ducts 312, respectively. Each of one or more inflation ducts 312 is configured to be fluidly connected to one or more inflation lumens 130, respectively, of catheter 100, such as to fluidly couple one or more fluid bags 314 to one or more inflation lumens 130, respectively. Thus, according to some embodiments, which are depicted in the figures, and wherein inflation lumens 130 include a pair of inflation lumens (i.e. first inflation lumen 130a and second inflation lumen 130b), inflation ducts 312 include a first inflation duct 312a and a second inflation duct 312b, and fluid bags 314 include a (pressable) first fluid bag 314a and a (pressable) second fluid bag 314b. First inflation duct 312a and second inflation duct 312b may be configured to be fluidly connected, at the distal ends (not numbered) thereof, to first inflation port 144a and second inflation port 144b, respectively. First fluid bag 314a and second fluid bag 314b are fluidly connected to the proximal end (not numbered) of first inflation duct 312a and to the proximal end (not numbered) of second inflation duct 312b, respectively.

According to some embodiments, the volume of first balloon 106, when fully inflated, may be about 10 mL (milliliter). According to some embodiments, the volume of second balloon 108, when fully inflated, may be between about 10 mL and about 20 mL.

Urine indicator 304 may include an elongated indicator tube 322 (urine indicator tube) and a container 324. Indicator tube 322 extends from an indicator tube distal tip 332 to an indicator tube proximal tip 334, which may be joined to container 324. Indicator tube 322 may be fluidly connected, e.g. at the proximal end (not numbered) thereof, to container 324, which may otherwise be fluidly sealed (optionally, controllably fluidly sealed). The function of urine indicator 304 is described below. Container 324 may be transparent, such as to allow seeing by eye when urine is present therein.

According to some embodiments, container 324 or indicator tube 322 may include a sensor configured to detect drainage of urine into container 324 or flow of urine via indicator tube 322. The sensor may be associated with an output device/means/interface configured to indicate (e.g. aurally and/or visually) to the user when urine drainage has been detected by the sensor.

According to some embodiments, urine indicator 304 is removably mounted (or mountable) on extractor 200, such that indictor tube 322 extends alongside one of extensions 212 and 214 (e.g. first extension 212), and distally projecting beyond that extension (e.g. first extension distal tip portion 242). When urine indicator 304 is mounted on extractor 200, container 324 may be positioned between handles 202 and 204. Indicator tube 322 may be dimensioned such that when urine indicator 304 is mounted on extractor 200, and extractor 200 is deployed within catheter 100 such that extension distal tip portions 242 and 244 are adjacent to first protrusion 152a and second protrusion 152b, respectively, indicator tube 322 extends through urine-expulsion valve 136 with urine indicator distal tip 332 being positioned, for example, between about 1 cm and about 2 cm distally to urine-expulsion valve 136.

According to some embodiments, balloon inflation system 302 is removably mounted (or mountable) on extractor 200. For example, with first fluid bag 314a and/or first inflation duct 312a mounted on first handle side-surface 256, and second fluid bag 314b and/or second inflation duct 312b mounted on second handle side-surface 258. According to some embodiments, not depicted in the figures, first handle 202 may include an internal passage and an opening on first handle distal edge 226 wherethrough first inflation duct 312a may be passed/set, and second handle 204 may include an internal passage and an opening on second handle distal edge 236 wherethrough second inflation duct 312b may be passed/set.

According to some embodiments, catheter kit 300 may be assembled prior to packaging thereof, so as to arrive assembled “out of the box”. More specifically, catheter kit 300 may arrive with extensions 212 and 214 inserted into urine-expulsion lumen 118, such that extension distal tip portions 242 and 244 are adjacent to first protrusion 152a and second protrusion 152b, respectively. Urine indicator 304 may arrive mounted on extractor 200, such that indicator tube 322 extends through urine-expulsion valve 136 (and urine indicator distal tip 332 is proximal to second balloon 106). Balloon inflation system 302 may arrive with first inflation duct 312a fluidly connected to first inflation port 144a and second inflation duct 312b fluidly connected to second inflation port 144b, thereby fluidly coupling first fluid bag 314a to first inflation lumen 130a and second fluid bag 314b to second inflation lumen 130b. First fluid bag 314a may include a marking (e.g. text printed thereon) indicating that first fluid bag 314a is fluidly coupled to first inflation lumen 130a and is intended to be used for inflating first balloon 106. Similarly, second fluid bag 314b may include a marking indicating that second fluid bag 314b is fluidly coupled to second inflation lumen 130b and is intended to be used for inflating second balloon 108. Optionally, balloon inflation system 302 may arrive mounted on/through extractor 200, as described above.

Each of fluid bags 314 may arrive filled with sterile water or a saline solution. According to some embodiments, each of inflation ducts 312 may include a respective (and breakable) seal (not shown) positioned at, or near, the proximal end of the inflation duct. The purpose of the seal is to maintain the fluids within fluids bags 314 until catheter 100 is deployed. Each of the seals may be configured to be broken when the respective fluid bag (from fluid bags 314) is squeezed, due to the pressure difference across the seal induced by the fluid, thereby allowing to force the fluid, via inflation ducts 312, into inflation lumens 130, as elaborated on below.

According to some embodiments, fluid bags 314 may be made of a material including rubber and/or silicone. According to some embodiments, indicator tube 322 may be made of a polymeric material, such as acrylonitrile butadiene styrene (ABS) plastic.

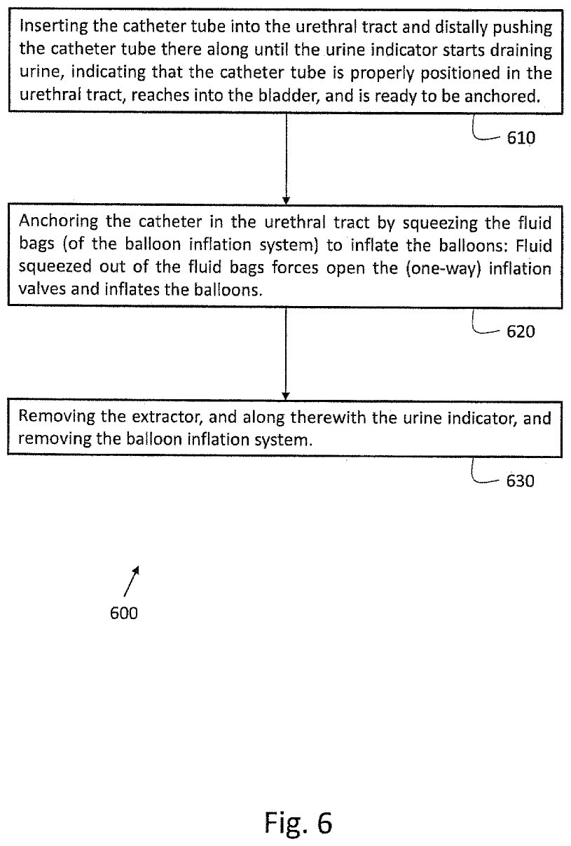

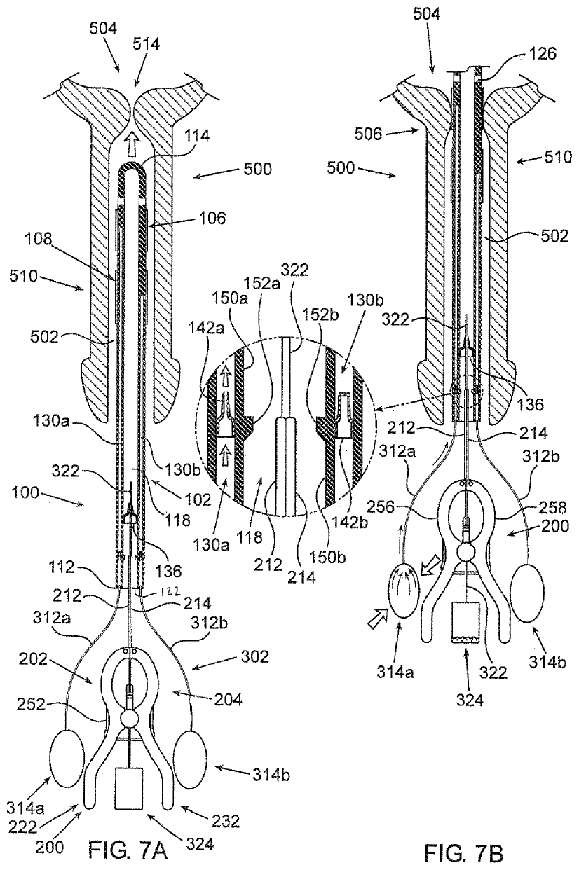

According to an aspect of some embodiments, there is provided a method 400 for removing (i.e. extracting) an indwelling urinary catheter, such as catheter 100, from a urethral tract of a male subject, using an extractor, such as extractor 200. FIG. 4 presents a flowchart of method 400, according to some embodiments. FIGS. 5A-5D schematically depict different stages in method 400, when implemented using catheter 100 and extractor 200, according to some embodiments.

According to some embodiments, method 400 includes:
A step 410 of deploying the extractor (e.g. extractor 200) in the catheter (e.g. catheter 100): When the extractor is in the first (closed) configuration, the extensions (e.g. extensions 212 and 214) of the extractor are inserted into the urine-expulsion lumen (e.g. urine-expulsion lumen 118), via the urine-expulsion port, such that the distal tip portions of the extensions are adjacent to protrusions (e.g. protrusions 152) in the urine-expulsion lumen.
A step 420 of opening the inflation valves (e.g. inflation valves 142), and thereby emptying the balloons (e.g. balloons 106 and 108): The extractor is switched from the first configuration to the second (open) configuration. The protrusions are forced by the distal tip portions of the extensions against the inflation valves, which are thereby opened, the fluid in the balloons flows out, and the balloons are emptied (or substantially emptied).
A step 430 of removing the extractor and the catheter: The extensions are pulled out (while still in the second configuration). As the extractor is (proximally) pulled, the distal tip portions of the extensions (e.g. extension distal tip portions 242 and 244) engage the protrusions, so that the proximal section of the catheter tube (e.g. tube proximal section 128) is dragged out together with the extensions. The rest of the catheter is then removed by pulling the proximal section of the catheter tube.

FIGS. 5A-5D present schematic views of catheter 100, extractor 200, and a penis 500 of a (man) subject, which combine front and cross-sectional views, according to some embodiments. More specifically, the views of catheter 100 and penis 500 are cross-sectional, while the view of extractor 200 is a front view. It is noted that the cross-sectional view of penis 500 is an anterior cross-sectional view.

According to some embodiments of catheter 100, and as depicted in the figures, protrusions 152 may slop outwardly in the proximal direction. Additionally or alternatively, according to some embodiments of catheter 100, protrusions 152 may be malleable such as to be impressed by/conform to the shape of (the engaging part) of extension distal tip portions 242 and 244, when extension distal tip portions 242 and 244 are forced against protrusions 152 in step 420, thereby potentially facilitating the gripping of protrusions 152 by extension distal tip portions 242 and 244 when catheter 100 is pulled out using extractor 200 in step 430.

According to some embodiments of catheter 100, inner side-surfaces 150 do not include protrusions 152. According to some such embodiments, in place of protrusions 152, each inner side-surfaces 150 includes a notch/groove positioned adjacently to inflation valves 142, respectively. Each notch may be dimensioned to accommodate the tip of one of extensions 212 and 214. It is noted that in embodiments wherein catheter 100 does not include protrusions 152, valves 142 may still be opened by pressing extension distal tip portions 242 and 244 against inner side-surfaces 150a and 150b, respectively, at a regions of inner side-surfaces 150a and 150b which are adjacent to inflation valves 142a and 142b, respectively. These regions may potentially be easier to locate when including the notches described above.

In FIG. 5A, extractor 200 is shown at the end of step 410, that is, deployed in catheter 100, which is secured within the urinary system of a male subject, according to some embodiments. More specifically, catheter 100 is shown inserted into penis 500 (i.e. into a urethral tract 502 of the penis), with first balloon 106 positioned in a bladder 504 (more precisely, in the bladder neck) of the subject, and with second balloon 108 anchored adjacently and proximally to a urinary sphincter 506 (i.e. on the proximal side of sphincter 506 and in the upper part of the urethra, in an expandable part thereof, which accommodates the second balloon) in a penile shaft 510 of the subject. Extractor 200 is in the first configuration (i.e. closed configuration) with first extension 212 and second extension 214 inserted into urine-expulsion lumen 118, such that first extension distal tip portion 242 and second extension distal tip portion 244 are positioned adjacently to, or substantially adjacently to, first protrusion 152a and second protrusion 152b, respectively.

Also indicated is a bladder neck 514 of the subject, which forms a lower part of bladder 504 and is positioned proximally to urinary sphincter 506.

FIG. 5B depicts extractor 200 deployed in catheter 100 during step 420: after extractor 200 has been switched to the second (open configuration) but before balloons 106 and 108 have been significantly emptied or emptied at all, according to some embodiments.

FIG. 5C depicts extractor 200 deployed in catheter 100 at the end of step 420, that is, after extractor 200 has been switched to the second configuration (i.e. open configuration), and after balloons 106 and 108 have been emptied (or substantially emptied), according to some embodiments. First inflation valve 142a and second inflation valve 142b are both open (as can be seen in the enlarged (closeup) view), so that balloons 106 and 108 are fluidly coupled to inflation ports 144 (e.g. first balloon 106 and second balloon 108 are fluidly coupled to first inflation port 144a and second inflation port 144b, respectively), and fluid (e.g. the sterile water or saline solution used to inflate the balloons) has drained out of balloons 106 and 108 (which are depicted deflated). During step 420, first inflation valve 142a may be kept open due to a lateral outwardly directed force (along the direction defined by the positive x axis) applied thereon by first protrusion 152a, resultant from the pressure exerted on first protrusion 152a by first extension distal tip portion 242. Similarly, second inflation valve 142b may be kept open due to a lateral outwardly directed force (along the direction defined by the negative x axis) applied thereon by second protrusion 152b, resultant from the pressure exerted on second protrusion 152b by second extension distal tip portion 244.

In FIG. 5D, catheter 100 and extractor 200 are shown during step 430, according to some embodiments: Extractor 200 is shown fully removed from urethral tract 502, and catheter 100 is shown partially removed from penis 500, with tube proximal section 128 pulled out of urethral tract 502, while the rest of catheter 100 is yet to be pulled out. More specifically, in FIG. 5D tube distal section 124 is shown no longer extending into bladder 504, so as to be fully positioned in urethral tract 502. Extractor 200 may be used to complete the removal of catheter 100, or once tube proximal section 128 has been pulled out, the rest of catheter 100 may be pulled out by gripping by hand tube proximal section 128 and pulling.

According to an aspect of some embodiments, there is provided a method 600 for deploying an indwelling urinary catheter, such as catheter 100, in the urinary system of a male subject, using a catheter kit, such as catheter kit 300. FIG. 6 presents a flowchart of method 600, according to some embodiments. FIGS. 7A-7E schematically depict stages in method 600, when implemented using catheter kit 300, according to some embodiments.

According to some embodiments, method 600 includes:
A step 610 of inserting the catheter into the urethral tract of the subject: With the catheter kit assembled (e.g. essentially as depicted in FIG. 3A), the catheter tube (e.g. catheter tube 102) is inserted via the urethral orifice and gradually (distally) pushed along the urethral tract until the urine indicator (e.g. urine indicator 304) starts draining urine, indicating that the distal section of the catheter tube (e.g. tube distal section 124) has reached the bladder, the first (distal) balloon (e.g. first balloon 106) is positioned in the bladder neck, and the second (proximal) balloon (e.g. second balloon 108) is positioned adjacently to the urinary sphincter on the proximal side of the urinary sphincter.
A step 620 of anchoring the catheter (i.e. securing/fixing the catheter within the urinary system) by inflating the balloons: The fluid bags (e.g. fluid bags 314) are pressed (squeezed). Fluid from the fluid bags forces open the (one-way) inflation valves (e.g. inflation valves 142), distally flows therethrough, and inflates the balloons.
A step 630 of removing the extractor (and thereby urine indicator 304) and the balloon inflation system (e.g. disconnecting inflation ducts 312 from inflation ports 144).

According to some embodiments, and as depicted in FIGS. 7A-7E, step 620 includes a sub-step 620a of inflating the first balloon, and a later sub-step 620b of inflating the second balloon (so that the first balloon is inflated before the second balloon). According to some such embodiments, the inflation of the first balloon prior to the inflation of the second balloon may help in preventing slippage of the second balloon during the inflation thereof, as some anchoring of the catheter is already provided the first balloon. It is noted that once the two balloons are inflated, the catheter is secured/fixed within the urinary system, with the first balloon preventing the catheter from being shifted/moving in the proximal direction (and potentially slipping out of the body) and the second balloon preventing the catheter from being shifted/moving in the distal direction (and potentially migrating into the bladder).

According to some embodiments, after step 630 is completed, the urine indicator may be unmounted (e.g. detached from extractor 200). If the balloon inflation system is also mounted on/in the extractor, then the balloon inflation system may also be unmounted.

The extractor may then be cleaned/sanitized, so as to be ready (if and when required) for removing the catheter from the urine system (as described in method 400). The balloon inflation system and urine indicator may then be discarded.

FIGS. 7A-7E present schematic views of catheter kit 300 and penis 500, which combine front and cross-sectional views, according to some embodiments. More specifically, the views of catheter 100 and penis 500 are cross-sectional, while the views of extractor 200, balloon inflation system 302, and urine indicator 304 are front views.

In FIG. 7A, catheter kit 300 is shown during step 610; that is, catheter kit 300 is assembled and catheter 100 is inserted into, and distally pushed along, urethral tract 502. In particular, extractor 200 is deployed in catheter 100 such that indicator tube 322 extends through urine-expulsion valve 136 (which is otherwise closed),

In FIG. 7B, catheter kit 300 is shown at the beginning of sub-step 620a of step 620, that is, at the beginning of the inflation of first balloon 106. Catheter kit 300 is assembled and catheter 100 is deployed in penis 500 (with second balloon 108 yet to be inflated). More specifically, first balloon 106 is positioned in bladder 504 (in bladder neck 514), and second balloon 108 is positioned in urethral tract 502, adjacently and proximally to urinary sphincter 506 and in an upper part of urethral tract, which is expandable and which accommodates second balloon 108. Inflation ducts 312a and 312b are fluidly connected to inflation ports 144a and 144b, respectively. Catheter 100 expands urinary sphincter 506 and urine that has drained, via openings 126, into urine-expulsion lumen 118, and therefrom, into indicator tube 322 and container 324, indicates catheter 100 has been properly deployed. First fluid bag 314a is being squeezed (as indicated by the arrows) so that fluid flowing out therefrom has forced, and maintains, first inflation valve 142a open, as indicated by the arrows in the close-up view.

In FIG. 7C, catheter kit 300 is shown at the beginning of sub-step 620b of step 620 and after sub-step 620a has ended, that is, at the beginning of the inflation of second balloon 108 and after first balloon 106 has been fully inflated. Second fluid bag 314b is being squeezed (as indicated by the arrows) so that fluid flowing out therefrom has forced, and maintains, second inflation valve 142b open, as indicated by the arrows in the close-up view. First balloon 106 is fully inflated, first fluid bag 314a is deflated, and first inflation valve 312a is closed again. More specifically, once a threshold amount of fluid (e.g. most of the fluid) has been forced out of first fluid bag 314a, the pressure that can be generated on the distal side of first inflation valve 312a is no longer sufficient to keep first inflation valve 312a open and first inflation valve 312a (automatically) closes. The same holds true with respect to second inflation valve 312b at the end of the inflation of second balloon 108, as described below.

According to some embodiments of catheter kit 300, first fluid bag 314a volume may be (i) sufficiently large to contain enough fluid, such as to allow fully, or substantially fully, inflating first balloon 106, but at the same time (ii) not too large so as to preclude forcing too much fluid into first balloon 106 with the result of over-pressurizing the fluid in first balloon 106, which may lead rupture thereof and/or damage to the bladder neck. Similarly, according to some embodiments, second fluid bag 314b volume may be (i) sufficiently large to contain enough fluid, such as to allow fully, or substantially fully, inflating second balloon 108, but at the same time (ii) not too large so as to preclude forcing too much fluid into second balloon 108 with the result of over-pressurizing the fluid in second balloon 108, which may lead to rupture thereof and/or damage to the bladder neck.

In FIG. 7D, catheter kit 300 is shown at the beginning of step 630, that is, at the beginning of the removal of extractor 200, balloon inflation system 302, and urine indicator 304. Catheter 100 is anchored (secured) in penis 500 (i.e. first balloon 106 is fully inflated and positioned in the bladder neck and second balloon 108 is fully inflated and anchored in urethral tract 502 proximally to urinary sphincter 506 such as to be adjacent thereto). Fluid bags 314a and 314b are deflated, and both of inflation valves 312a and 312b are closed.

In FIG. 7E, catheter kit 300 is shown at the end of step 630, that is, after extractor 200, balloon inflation system 302, and urine indicator 304 have been removed. Catheter 100 is ready for use.

In order to urinate, a subject—with catheter 100 deployed in the urinary system thereof, as described above—will press the penile shaft 510 between e.g. a thumb and forefinger, such as to squeeze urine-expulsion valve 136 in accordance with FIG. 1E (i.e. in accordance with arrows F₁ and F₂ in FIG. 1E), thereby opening urine-expulsion valve 136 and allowing urine to flow out. Thus, if catheter 100 is deployed as depicted in FIG. 7E, urine-expulsion valve 136 may be opened by pressing on the anterior of penile shaft 510 and in the direction of the negative y-axis, and simultaneously pressing on the posterior of penile shaft 510 in the direction of the positive y-axis, such that e.g. the pressing thumb and index finger are positioned on the two sides of urine-expulsion valve 136. According to some embodiments, circumferential base 140 may be elongated to ensure that most of the forces exerted by the thumb and index finger are in fact directed to circumferential base 140. According to some embodiments, the width W of circumferential base may be as large as about 1 cm, about 1.5 cm, or even about 2 cm. Each possibility corresponds to separate embodiments. According to some embodiments, circumferential base 140 may extend also in the proximal direction, such as to surround at least a distal portion of beak 170. According to some embodiments, urine-expulsion valve 136 is configured to automatically shut, once the pressing on penile shaft 510 is ceased/sufficiently relaxed, thereby ensuring that no urine leaks out once more urine is produced. According to some embodiments, to close urine-expulsion valve 136, the sides of penile shaft 510 must be pressed, such as to induce squeezing of urine-expulsion valve 136 in accordance with FIG. 1D (i.e. in accordance with arrows F₃ and F₄ in FIG. 1D).

Although catheter kit 300 has been described with extractor 200 being used not only in the removal of catheter 100 from the urinary system but also in the deployment of catheter 100 therein, it will be understood that the scope of the disclosure also covers the case wherein extractor 200 is not intended to be used in the deployment of catheter 100, with catheter kit 300 additionally or alternatively including an applicator for the deployment of catheter 100. Thus, according to an aspect of some embodiments, there is provided a catheter kit. The catheter kit includes catheter 100, an applicator, and, optionally, extractor 200. The applicator includes an applicator body configured to be held by hand. The applicator body may be similar in shape to the aggregate of handles 202 and 204 of extractor 200, but, in contrast, need not include any movable parts. For example, the applicator body may be formed of a single piece. The applicator further includes a balloon inflation system, which may be similar to balloon inflation system 302, and a urine indicator, which may be similar to urine indicator 304. In particular, the mounting of the balloon inflation system and the urine indicator on the applicator body may be similar to the mounting of balloon inflation system 302 and urine indicator 304 on extractor 200. Similarly to catheter kit 300, the catheter kit may arrive assembled with an indicator tube of the urine indicator inserted into catheter tube 102 and through urine-expulsion valve 136, and the distal ends of inflation ducts of the balloon inflation system being connected to/fitted into inflation ports 144.

As used herein, according to some embodiments, the terms “anchor”, “secure”, and “fix”—with reference to the deployment of a catheter (such as the catheters disclosed herein) inside the body of a subject—are interchangeable.

As used herein, according to some embodiments, the terms “endo-urethral catheter” and “indwelling urinary catheter” are used interchangeably.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. An endo-urethral catheter for a male subject, the catheter comprising an elongated catheter tube, an inflatable first balloon, and an inflatable second balloon, wherein the catheter tube comprises:
a urine-expulsion lumen extending along a length of the catheter from a tube proximal end to a tube distal section;
one or more openings positioned on the tube distal section and fluidly connected to the urine-expulsion lumen; and
a urine-expulsion valve positioned in the urine-expulsion lumen;
wherein the first balloon is disposed about the tube distal section proximally to the one or more openings, and wherein the second balloon is positioned proximally to the first balloon and distally to the urine-expulsion valve;
wherein the catheter is configured to be deployed in a urinary system of a male subject with the balloons inflated, such that (i) the one or more openings fluidly couple the urine-expulsion lumen to a bladder of the subject, (ii) the first balloon is positioned in a bladder neck of the subject, and (iii) the second balloon is positioned in a urethral tract of the subject, adjacently and proximally to a urinary sphincter of the subject, the first balloon and the second balloon thereby anchoring the catheter within the urinary system; and
wherein the urine-expulsion valve is configured to be controllably opened and shut, such as to respectively fluidly couple and decouple the bladder to the tube proximal end, thereby allowing the subject to controllably expel urine.

2. The endo-urethral catheter of claim 1, wherein, when the catheter is properly deployed, the tube proximal end is positioned within the urethral tract.

3. The endo-urethral catheter of claim 1, wherein the first balloon is configured to prevent the catheter from slipping out of the body of the subject, when the catheter is properly deployed with the first balloon being inflated in the bladder neck.

4. The endo-urethral catheter of claim 1, wherein the second balloon is configured to prevent distal migration of the catheter along the urethral tract, when the catheter is properly deployed with the second balloon inflated and positioned in the urethral tract, adjacently and proximally to the urinary sphincter.

5. The endo-urethral catheter of claim 1, wherein the urine-expulsion valve is configured (i) to be opened by squeezing thereof, and (ii) to be automatically shut when the squeezing is ceased/sufficiently relaxed, being thereby configured to prevent involuntary leakage of urine, wherein the urine-expulsion valve is configured to function as a one-way valve preventing proximal flow of fluid therethrough and wherein the urine-expulsion valve is a duckbill valve.

6. (canceled)

7. (canceled)

8. The endo-urethral catheter of claim 5, wherein the urine-expulsion valve is configured to be opened by pressing a penile shaft and/or a glans penis of the subject, such as to squeeze the urine-expulsion valve, when the catheter is properly deployed.

9. The endo-urethral catheter of claim 1, wherein the catheter comprises at least one inflation lumen extending in parallel to the urine-expulsion lumen in the distal direction from at least one inflation port positioned at the tube proximal end, the at least one inflation lumen being configured to inflate the balloons and wherein each of the at least one inflation lumen comprises one or more inflation valves configured to prevent fluid flow in the proximal direction, and which, when open, fluidly couple the balloons to the at least one inflation port, and, when closed, fluidly decouple the balloons from the at least one inflation port, thereby acting to maintain the balloons inflated.

10. (canceled)

11. The endo-urethral catheter of claim 9, wherein each of the one or more inflation valves is positioned proximally to the urine-expulsion valve.

12. The endo-urethral catheter of claim 9, wherein the at least one inflation lumen comprises a first inflation lumen and a second inflation lumen extending inside tube walls of the catheter tube on opposite sides of the urine-expulsion lumen and wherein the first inflation lumen is fluidly connected to the first balloon and the second inflation lumen is fluidly connected to the second balloon.

13. (canceled)

14. The endo-urethral catheter of claim 12, wherein the urine-expulsion lumen comprises a first inner side-surface on a first section of the tube walls separating the urine-expulsion lumen from the first inflation lumen, and a second inner side-surface on a second section of the tube walls opposite to the first section;
wherein the first inner side-surface comprises a first protrusion extending laterally therefrom and positioned adjacently to the first inflation valve, and the second inner side-surface comprises a second protrusion extending laterally therefrom and positioned adjacently to the second inflation valve; and
wherein the first inflation valve and the first protrusion are configured such as to allow opening the first inflation valve by pressing on the first protrusion, and the second inflation valve and the second protrusion are configured such as to allow opening the second inflation valve by pressing on the second protrusion.

15. The endo-urethral catheter of claim 14, wherein the protrusions are malleable.

16. The endo-urethral catheter of claim 14, wherein each of the first inflation valve and the second inflation valve is configured to function as a one-way valve preventing proximal flow of fluid therethrough.

17. The endo-urethral catheter of claim 16, wherein each of the first inflation valve and the second inflation valve is a duckbill valve.

18. The endo-urethral catheter of claim 9, wherein the first balloon and the second balloon are fluidly connected.

19. An extractor for removing an endo-urethral catheter deployed in a urine system of a male subject, the extractor comprising:
a first handle comprising a first proximal section and a first distal section which comprises a first distal edge, and a second handle comprising a second proximal section and a second distal section which comprises a second distal edge, the handles being configured to allow pressing the proximal sections towards one another and thereby distancing the distal edges from one another;
a first extension, and second extension, each in the form of an elongated rod;
wherein the first extension distally extends from the first distal edge, and the second extension distally extends from the second distal edge; and
wherein the extractor is configured to be switched between at least two configurations:
a closed configuration, wherein the extensions are disposed alongside one another, such as to contact, or nearly contact, one another; and
an open configuration, wherein the proximal sections are pressed towards one another and the distal tip portions of the extensions are spaced apart from one another.

20. The extractor of claim 19, wherein in the open configuration the extensions are arranged in a V-like configuration.

21. (canceled)

22. (canceled)

23. (canceled)

24. (canceled)

25. (canceled)

26. (canceled)

27. A catheter kit comprising a catheter, according to claim 1, an extractor, a balloon inflation system, and a urine indicator;
wherein the extractor comprising:
a first handle comprising a first proximal section and a first distal section which comprises a first distal edge, and a second handle comprising a second proximal section and a second distal section which comprises a second distal edge, the handles being configured to allow pressing the proximal sections towards one another and thereby distancing the distal edges from one another;
a first extension, and second extension, each in the form of an elongated rod;
wherein the first extension distally extends from the first distal edge, and the second extension distally extends from the second distal edge; and
wherein the extractor is configured to be switched between at least two configurations:
a closed configuration, wherein the extensions are disposed alongside one another, such as to contact, or nearly contact, one another; and
an open configuration, wherein the proximal sections are pressed towards one another and the distal tip portions of the extensions are spaced apart from one another,
wherein the balloon inflation system comprises (i) a pressable first fluid bag and a first inflation duct configured for fluidly coupling the first fluid bag to the first inflation lumen, and (ii) a pressable second fluid bag and a second inflation duct configured for fluidly coupling the second fluid bag to the second inflation lumen; and
wherein the urine indicator includes a urine indicator tube fluidly connected to, and extending distally from, a container, the urine indicator tube being removably attached to the extractor such as to be aligned with the extensions, and to extend in the distal direction beyond the extensions, when the extractor is in the closed configuration.

28. The catheter kit of claim 27, wherein the catheter kit is assembled with the extensions of the extractor, which is in the closed configuration, inserted into the urine-expulsion lumen such that the distal tip portions of the extensions are adjacent to the protrusions on the inner side-surfaces and such that the urine indicator tube extends through the urine-expulsion valve; and
wherein the inflation ducts of the balloon inflation system are fluidly connected to the inflation ports of the catheter tube.

29. A method for removing a catheter, according to claim 1, which is deployed in a urinary system of a male subject, using an extractor, wherein the catheter is deployed such that the first balloon is inflated and positioned at a bladder neck of the urinary system, and the second balloon is inflated and positioned in a urethral tract of the urinary system, adjacently and proximally to a urinary sphincter of the urinary system, such that the balloons secure the catheter within the urinary system, the method comprising:
deploying the extractor, when in the closed configuration, in the catheter, by inserting the extensions of the extractor into the urine-expulsion lumen, such that the distal tip portions of the extensions are adjacent to the protrusions in the urine-expulsion lumen;
emptying the balloons by switching the extractor to the open configuration, thereby pressing on the protrusions and opening the inflation valves; and
pulling out the proximal section of the catheter tube by pulling out the extractor, and then pulling out the rest of the catheter tube.

30. A method for deploying a catheter in a urinary system of a male subject using a catheter kit, according to claim 27, the method comprising:
inserting the catheter into a urethral tract of the urine system and gradually pushing the catheter there along until the urine indicator starts draining urine, indicating that the distal section of the catheter tube has reached the bladder, the first balloon is positioned in a bladder neck of the urine system, and the second balloon is positioned in the urethral tract, adjacently and proximally to a urinary sphincter of the urine system;
inflating the balloons to anchor the catheter by squeezing the fluid bags of the balloon inflation system, so that fluid ejected from the fluid bags forces open the inflation valves in the inflation lumens, distally flows therethrough, and inflates the balloons; and
removing the extractor, and thereby the urine indicator, and removing the balloon inflation system.

31. (canceled)
